(19) United States
(12) Patent Application Publication
(10) Pub. No.: US 2022/0395547 A1
(43) Pub. Date: Dec. 15, 2022

(54) LIPOSOMAL FORMULATIONS FOR DELIVERY OF CANNABINOIDS AND METHODS OF MAKING THEREOF

(71) Applicant: Vella Bioscience, Inc., Worcester, MA (US)

(72) Inventors: Michael Frid, Medford, MA (US); Harin Padma-Nathan, Los Angeles, CA (US)

(21) Appl. No.: 17/775,288

(22) PCT Filed: Nov. 3, 2020
(86) PCT No.: PCT/US20/58719

§ 371 Date: Jul. 6, 2022

Related U.S. Application Data
(60) Provisional application No. 62/932,754, filed on Nov. 8, 2019.

(51) Int. Cl. A61K 36/185; A61K 9/16; A61K 31/352; A61K 47/22; A61K 47/36

(52) U.S. Cl. CPC A61K 36/185; A61K 9/1617; A61K 31/352; A61K 47/22; A61K 47/36

(57) ABSTRACT

Methods for producing large cannabinoid-loaded liposomes are provided. In one example, these liposomes are large and mostly multilamellar. In another example, the liposomes are a combination of large multilamellar and unilamellar liposomes.

### PRIORITY

This application claims priority to the U.S. Provisional Patent Application No. 62/932,754, filed on Nov. 8, 2019, which is hereby incorporated by reference in its entirety.

### FIELD OF THE INVENTION

The present disclosure relates to liposomal formulations for delivery of highly lipophilic compounds, for example, such as cannabinoids.

### BACKGROUND

Liposomes have been used for delivery of active pharmaceutical ingredients (APIs) and nutraceuticals for several decades. Liposomes are classified by size, from large of >20 μm in size to small of below 100 nm; by lamellarity, multilamellar and unilamellar; by surface properties, charged, uncharged, or PEGylated; and stabilized or edge-activated bilayers. These differences translate into widely tunable properties such as payload, circulation time, and interaction with cells. Therefore, liposomes offer myriad options and, consequently, flexibility in designing a delivery system adapted for a specific payload and delivery route.

Wider adoption of liposomal delivery systems, especially outside of pharmaceutical drug delivery applications, has been slowed by, among other issues, the difficulty in large scale manufacturing of liposomes. The classical thin layer hydration method is straightforward, requires inexpensive equipment widely available in chemistry laboratories, and is easy to implement on small scale (e.g., 50 to 500 mg) and therefore this method is universally used for research yet is unsuitable for commercial scale up.

A variety of methods have been developed for the production of liposomes (Laouini, A., et al. (2012). “Preparation, Characterization and Applications of Liposomes: State of the Art.” Journal of Colloid Science and Biotechnology 1(2): 147-168.), yet almost all methods require at least one additional step to remove organic solvent either by direct evaporation, for example if using a water-immiscible solvent (diethyl ether), via rotary evaporation, or via freeze drying or lyophilization, or by dialysis through a size exclusion membrane after hydration. Alternatively, mixtures of water and water miscible-solvents may be removed by several methods, such as freeze drying or lyophilization, or spray drying to produce dehydrated liposomes, sometimes called pre-liposomes or pro-liposomes (proliposomes), for subsequent reconstitution post-manufacturing or prior to use. Solvent removal is time consuming and costly, whether solvent is recovered or must be disposed of, and requires significant investment in production equipment, yet each of the abovementioned methods has its advantages.

Alternatively, methods have been devised to yield liposomal preparations that contain the organic solvents to yield the so-called ethosomes, glycerosomes, and PG-liposomes. Most of these systems require the addition of the organic solvents to proliposomes during hydration step but some methods allow introduction of organic solvents as solvents for the lipid components. If these liposomal preparations contain low enough absolute quantities and concentrations of organic solvents compatible with the intended route of delivery, and if the properties of the liposomes prepared by such a method, such as size, lamellarity, stability, loading of active, etc. are adequate, then no further preparation steps would be required thus saving considerable resources in manufacturing.

Different routes of systemic administration are attracting increased attention, such as delivery through various body mucosae, for example through the skin (transdermal), through buccal, sublingual, and vaginal tissues, as well as non-systemic delivery designed to produce localized rather than whole-body effects.

A particular challenge for liposomal delivery is presented by cannabinoids. Delivery of cannabinoids, as exemplified by cannabidiol (CBD) and Δ⁹-tetrahydrocannabinol (THC), whether systemic or to certain tissues or compartments, is impeded because of the extremely high lipophilicity, the experimentally determined log P or calculated c log P values for some cannabinoids are estimated at 8 or higher, and strong first pass effect. Thus, there has been increasing interest in developing new and/or improved methods for delivery of cannabinoids by routes other than the traditional ingestion or inhalation, especially in light of the realization that “vaping”, as practiced currently, may be unsafe.

Certain liposomal formulations for delivery of cannabinoids have been published. For example, U.S. Pat. No. 9,095,555 describes the preparation of cannabinoid-containing liposomes by the ethanol injection method, a two-step (at least) process whereby a solution of phospholipid, cannabinoid, and other ethanol soluble ingredients is injected into water or aqueous buffer, followed by solvent removal by various means, such as evaporation, freeze drying, or spray drying. As another example, U.S. Pat. No. 5,716,638 describes the preparation of cannabinoid-containing ethosomes for topical and transdermal delivery containing a high proportion of C₂-C₄ organic solvents, such as ethanol (20-50%) or a mixture of ethanol and propylene glycol (22-70%), such high concentration of solvents needed to aide permeation through human skin. US Pat. App. Publication No. 2018/0360757 describes a multi-step preparation of complex multilamellar vesicles containing CBD for topical and transdermal delivery for treatment or alleviation of pain and irritation, however, in all exemplified embodiments the final formulation contains complex mixtures of ingredients including cholesterol and potentially irritating or allergic reaction causing excipients, such as benzalkonium chloride, parabens, and Cremophor EL (Kolliphor® EL).

Therefore, there is a need for more effective formulations of cannabinoids tailored to different administration routes and to the needs of patients and users, and for such formulations to be manufacturable with reasonable cost and capital expenditures.

### DETAILED DESCRIPTION OF THE FIGURES

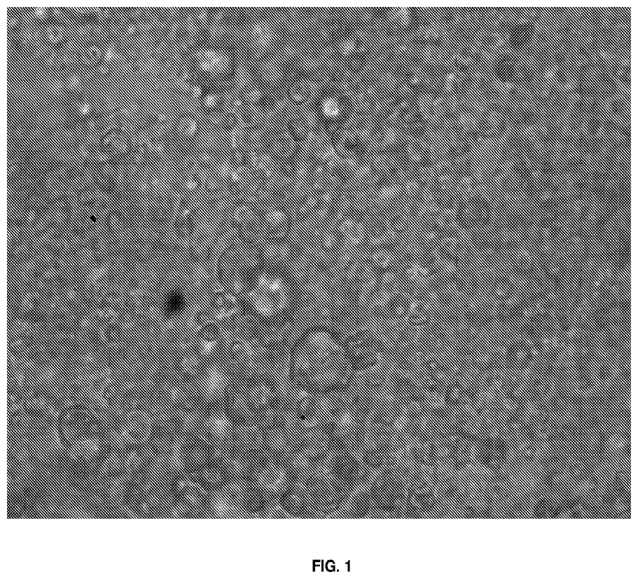

FIG. 1 shows a sample of lotion prepared as described in Example 10 was withdrawn after the formulation equilibrated to ambient temperature and was placed, undiluted, onto a borosilicate glass microscope slide, covered with a coverslip, and examined using an upright microscope and a 40× objective.

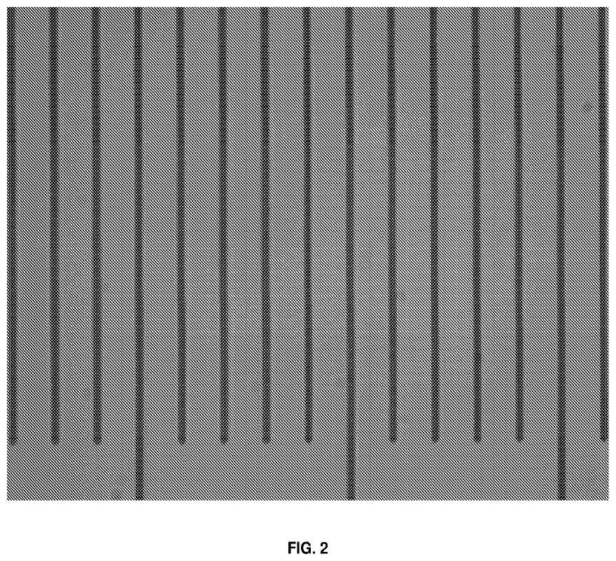

FIG. 2 shows 0.01 mm micrometer image acquired using an upright microscope and a 40× objective.

### SUMMARY OF THE INVENTION

In the first aspect, the invention features methods for producing large cannabinoid-loaded liposomes. In one embodiment, these liposomes are large and mostly multilamellar. In another embodiment, the liposomes are a combination of large multilamellar and unilamellar liposomes.

In another aspect, the invention features a scalable, highly simplified “one-pot” method for manufacturing cannabinoid-loaded liposomes; “one-pot” because all of the ingredients are mixed in a single vessel with no subsequent transfers to other vessels or reactors for addition of other ingredients or follow up treatment. In one embodiment, this method involves dissolution or dispersion of phospholipid(s) and cannabinoid(s) in propylene glycol or glycerin, or another water-miscible physiologically-acceptable solvent or a mixture of such solvents, followed by addition of this monophasic mixture to an aqueous phase to form a liposomal suspension with a relatively low fraction of non-aqueous solvent. In another embodiment, the method involves creating liposomal suspension in a mixture of glycerin and water, which mixture contains phospholipid(s), cannabinoid(s), and other ingredients, with a relatively low fraction of non-aqueous solvent.

In particular, the invention provides a method for a “one-pot” preparation of a cannabinoid liposome gel, lotion, or cream, which preparation does not require sophisticated manufacturing equipment such as freeze dryers, lyophilizers, or spray dryers. Specifically, in one embodiment, the invention is a “one-pot” method for the preparation of a suspension formulation of one or more cannabinoids in large liposomes, wherein the addition of a pre-heated homogenous mixture of phospholipids (e.g., SPC, HSPC, and DSPC), and cannabinoid(s) (described below) in a water-miscible solvent (e.g., propylene glycol, glycerin, or a mixture thereof) to a pre-heated aqueous solution under stirring, followed by stirring in a manner and for a period of time such that a homogenous suspension is produced. In certain embodiments, such as in the case of a lubricating agent (e.g., hydroxyethyl cellulose and or hyaluronic acid) is added to the mixture.

In certain other embodiments, stabilizers, for example, ascorbic acid (vitamin C) and sodium ascorbate are added to the mixture. In certain other embodiments, a lotion, or cream-like, composition is produced by adding a thickening agent (e.g., polyacrylate crosspolymer-6).

In yet another aspect, the liposomal formulation is compatible with polyurethane, latex or polyisoprene condoms. In certain embodiments, the composition is suitable for administering topically to skin, mucosal areas, including genital areas, used as a lubricant, and can be administered intravaginally, rectally, onto oral mucosa, or may be ingested (for example, when formulated into dissolvable capsules, or as food or beverage). When desirable, preservatives can be added to the formulations, particularly, when packaged for multiple uses.

A more detailed description of the invention follows.

### DETAILED DESCRIPTION OF THE INVENTION

The invention provides a scalable, highly simplified method for manufacturing cannabinoid-loaded liposomal formulations that can be used in a variety of ways.

As used herein, the term “cannabinoid” or “cannabinoids” refer to phytocannabinoids produced, in whatever quantity, by plants Cannabis sativa and Cannabis indica, which naturally contain different amounts of the individual cannabinoids (Elsohly, M. A. and D. Slade (2005). “Chemical constituents of marijuana: the complex mixture of natural cannabinoids.” Life Sciences 78(5): 539-548), and to synthetic analogues of phytocannabinoids, which compounds may be manufactured by isolation from Cannabis plants and chemovars thereof, by using yeast or other means utilizing biotechnology, by chemical synthesis, by combination of these methods, or by any other means. The terms “cannabinoid” or “cannabinoids” refer to compounds having log P or c log P or ≥4, wherein log P is an n-octanol/water partition coefficient obtained experimentally or calculated (c log P) by methods known to those skilled in the art. The terms “cannabinoid” or “cannabinoids” refer, therefore, for example, to (−)-trans-Δ⁹-tetrahydrocannabinol (Δ⁹-THC or THC), Δ⁸-tetrahydrocannabinol (Δ⁸-THC), (−)-trans-cannabidiol (CBD), cannabinol (CBN), cannabichromene (CBC), cannabicyclol (CBL), cannabielsoin (CBE), cannabinoldiol, cannabitriol, cannabigerol (CBG), cannabifuran (CBF), and their homologues containing a propyl rather than a pentyl side chain, such as cannabidivarin (CBDV), cannabivarin (CBV or cannabivarol), tetrahydrocannabivarin (THCV or THV), cannabichromene propyl analogue, as well as nabilone (racemic mixture or mixture of individual enantiomers in whatever stereoisomeric excess or purity), levonantradol (CP 50,556-1), cannabilactone (AM-1714), cannabicyclohexanol ((C8)-CP 47,497), (C9)-CP 47,497, AM-2389, AM-4030, AM-4056, (−)-HU-210, (+)-HU-210, racemic HU-210 or a mixture of individual enantiomers in whatever stereoisomeric excess or purity, ajulemic acid (HU-239), HU-243, HU-308, HU-320, HU-331, HU-336, HU-345, 11-hydroxy-Δ⁹-tetrahydrocannabinol (11-OH-THC), 11-carboxy-Δ⁹-tetrahydrocannabinol (11-CO₂H-THC), 7-hydroxycannabidiol (7-OH-CBD), 7-carboxycannabidiol (7-OH-CBD), CP 55,940, JWH-133, AM-087, AM-356, AM-404, AM-678, AM-855, AM-905, AM-906, AM-919, AM-938, CP 47,497, (C6)-CP 47,497, (C7)-CP 47,497, CP 55,940, AMG-36, AMG-41, KM-233, JWH-051, JWH-102, JWH-056, JWH-057, JWH-065, JWH-103, JWH-133, JWH-139, JWH-142, JWH-143, JWH-161, JWH-186, JWH-187, JWH-188, JWH-190, JWH-191, JWH-215, JWH-216, JWH-217, JWH-224, JWH-225, JWH-226, JWH-227, JWH-229, JWH-230, JWH-233, JWH-247, JWH-254, JWH-256, JWH-277, JWH-278, JWH-298, JWH-299, JWH-300, JWH-301, JWH-310, JWH-336, JWH-338, JWH-339, JWH-340, JWH-341, JWH-349, JWH-350, JWH-352, JWH-353, JWH-354, JWH-355, JWH-356, JWH-357, JWH-358, JWH-359, JWH-360, JWH-361, JWH-362, 0-1871, and any combination of two or more of these compounds. Cannabinoids may be isolated from plants as mixtures of cannabinoids and other plant-derived materials, such as terpenes, flavonoids, etc. or cannabinoids may be purified substances, and may be amorphous or exist in one or more different crystalline states (polymorphs).

The concentration of a cannabinoid, or a mixture of two or more cannabinoids, in a formulation may be approximately 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, 31 mg/mL, 32 mg/mL, 33 mg/mL, 34 mg/mL, 35 mg/mL, 36 mg/mL, 37 mg/mL, 38 mg/mL, 39 mg/mL, or 40 mg/mL. Alternatively, the concentration of a cannabinoid, or a mixture of two or more cannabinoids, in a formulation may be 1 mg/g, 2 mg/g, 3 mg/g, 4 mg/g, 5 mg/g, 6 mg/g, 7 mg/g, 8 mg/g, 9 mg/g, 10 mg/g, 11 mg/g, 12 mg/g, 13 mg/g, 14 mg/g, 15 mg/g, 16 mg/g, 17 mg/g, 18 mg/g, 19 mg/g, 20 mg/g, 21 mg/g, 22 mg/g, 23 mg/g, 24 mg/g, 25 mg/g, 26 mg/g, 27 mg/g, 28 mg/g, 29 mg/g, 30 mg/g, 31 mg/g, 32 mg/g, 33 mg/g, 34 mg/g, 35 mg/g, 36 mg/g, 37 mg/g, 38 mg/g, 39 mg/g, or 40 mg/g. Alternatively, a cannabinoid or a mixture of cannabinoids may be present in a weight to weight (w/w) ratio relative to phospholipid of 1/20, 1/19, 1/18, 1/17, 1/16, 1/15, 1/14, 1/13, 1/12, 1/11, 1/10, 1/9, 1/8, 1/7, 1/6, or 1/5.

In preferred embodiments, the cannabinoids are Δ⁹-THC, Δ⁸-THC, CBD, and CBN or mixtures thereof. In most preferred embodiments, the cannabinoids are selected one or both of THC and CBD. In some embodiments, the cannabinoid is CBD (for example, cannabis-derived CBD or hemp-derived CBD. In some embodiments, the CBD is hemp-derived and contains less than 0.3% THC.

As used herein, the term “phospholipid” or “phospholipids” refers to amphiphilic compounds comprising at least one saturated or unsaturated hydrophobic fatty acid moiety and a hydrophilic moiety comprising a phosphate group. These include, for example, dicetyl phosphate, soya phosphatidylcholine (SPC), egg phosphatidylcholine (EPC), hydrogenated soya phosphatidylcholine (HSPC), soya lecithin, hydrogenated soya lecithin, sphingomyelin, dioleoyl phosphatidylcholine (DOPC), dilinoleoyl phosphatidylcholine (DLPC), dioleoyl phosphatidylethanolamine (DOPE), dimyristoyl phosphatidylethanolamine (DMPE), dipalmitoyl phosphatidylethanolamine (DPPE), dimyristoyl phosphatidylcholine (DMPC), dipalmitoyl phosphatidylcholine (DPPC), distearoyl phosphatidylcholine (DSPC), dilauroyl phosphatidylcholine (DLPC), 1-myristoyl-2-palmitoyl phosphatidylcholine, 1-palmitoyl-2-myristoyl phosphatidylcholine, 1-palmitoyl phosphatidylcholine, 1-stearoyl-2-palmitoyl phosphatidylcholine, dipalmitoyl sphingomyelin, distearoyl sphingomyelin, soya phosphatidylinositol (SPI), hydrogenated phosphatidylinositol (HPI), dimyristoyl phosphatidylglycerol (DMPG), dipalmitoyl phosphatidylglycerol (DPPG), distearoyl phosphatidylglycerol (DSPG), dimyristoyl phosphatidic acid (DMPA), dipalmitoyl phosphatidic acid (DPPA), dimyristoyl phosphatidylserine (DMPS), dipalmitoyl phosphatidylserine (DPPS), hydrogenated soya phosphatidylglycerol, dioleoyl phosphatidylglycerol (DOPG), distearoyl phosphatidic acid (DSPA), and mixtures thereof, and salts thereof, preferably sodium or ammonium salts. Phospholipids may be present, on weight-to-weight (w/w) basis relative to total weight of a composition, at a level of 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, or 25%. In preferred embodiments, the phospholipid is one of or is a combination of two or more of SPC, EPC, HSPC, or DSPC. In general, it may be desirable that to employ phospholipids are condom-compatible. In certain embodiments, the liposome constituent lipids do not include cholesterol or its derivatives. In some embodiments, the lipids consist of, or consist essentially of, of the phospholipids recited above, or a subset thereof.

As used herein, the term “cryoprotectant” or “cryoprotectants” or “bulking agent” or “bulking agents” refers to compounds such as, for example, mannitol, sorbitol, lactose, trehalose, sucrose, dextran of different molecular weights such as dextran 40, inulin, glycine, L-arginine, α-cyclodextrin, β-cyclodextrin, γ-cyclodextrin, hydroxypropyl-β-cyclodextrin, hydroxypropyl-γ-cyclodextrin, randomly methylated-β-cyclodextrin, sulfobutyl ether β-cyclodextrin (SBE β-CD), hydroxypropyl methylcellulose (HPMC, hypromellose), methylcellulose, polyvinylpyrrolidone (PVP) K15, K16-18, K30, or K90, citric acid, sodium citrate, poloxamer 188 (Pluronic® F-68), poloxamer 407 (Pluronic® F-127), or polyvinyl caprolactam-polyvinyl acetate-polyethylene glycol graft copolymer (Soluplus®).

As used herein, the term “stabilizer” or “stabilizers” refers to, for example, ascorbic acid, ascorbate salts such as sodium or potassium ascorbate, citric acid, citrate salts such as, for example, sodium or potassium citrate, ethylenediaminetetraacetic acid (EDTA), ETDA salts such disodium EDTA, dipotassium EDTA, trisodium EDTA, tetrasodium EDTA, or calcium disodium EDTA, hydroxyethyl ethylenediamine triacetic acid (HEDTA), trisodium HEDTA, diethylenetriaminepentaacetic acid (DTPA), ethylenediamine-N,N′-disuccinic acid (EDDS), trisodium EDDS, DTPA pentasodium salt (pentasodium diethylenetriaminepentaacetate), methylglycinediacetic acid, trisodium dicarboxymethyl alaninate, d-glucono-1,5-lactone, gluconic acid and its salts such as sodium or potassium gluconate, or calcium gluconate, iminodisuccinic acid tetrasodium salt (tetrasodium iminodisuccinate), α-tocopherol, α-tocopherol acetate, ascorbyl palmitate, ascorbyl stearate, butylated hydroxytoluene (BHT), or butylated hydroxyanisole (BHA).

As used herein, the term “water-miscible solvent”, “water-miscible solvents”, “water-soluble solvent”, or “water-soluble solvents” refers to compounds such as, for example, ethyl alcohol (ethanol), t-butyl alcohol (t-butanol, tert-butanol, or TBA), polyethylene glycols (PEGs or macrogols) of different molecular weights such as PEG 300, PEG 400, PEG 600, PEG 1500, glycerin, diethylene glycol monoethyl ether (Transcutol®, diethylene glycol ethyl ether or 2-(2-ethoxyethoxy)ethanol), triacetin (glycerin triacetate), and propylene glycol (PG), which solvents may be used alone or as a combination of two or more solvents, with water-miscible solvents comprising, on weight-to-weight (w/w) basis relative to total weight of a formulation of 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, or 20%. In general, the compositions of the invention contain no more than 20% of PG, no more than 20% of glycerin, and no more than 20% of both PG and glycerin when both are present. Preferably, the compositions of the invention contain 6-20%, 8-18%, 6-16%, 6-14%, 8-16%, 8-14%, or 8-12% of PG. Likewise, the compositions of the invention contain 6-20%, 8-18%, 6-16%, 6-14%, 8-16%, 8-14%, or 8-12% of glycerin.

As user herein, the term “antimicrobial agent”, or “antimicrobial agents”, or “antimicrobial”, or “antimicrobials”, or “preservative”, or “preservatives” refers to substances that inhibit growth or kill microorganisms, whether antibacterial and/or antifungal agents, such as, for example, methyl paraben (methylparaben), ethyl paraben (ethylparaben), propyl paraben (propylparaben), butyl paraben (butylparaben), and heptyl paraben (heptylparaben), benzoic acid and benzoic acid salts such as sodium benzoate, dehydroacetic acid and sodium dehydroacetate, sorbic acid and its salts such as sodium sorbate, salicylic acid and its salts such as sodium salicylate, p-anisic acid, caprylhydroxamic acid, caprylic acid and its salts such as sodium caprate, levulinic acid and its salts such as sodium levulinate, undecylenic (10-undecenoic) acid and its salts such as sodium undecylenate, eugenol, menthol, 1,2-pentanediol, 1,2-hexanediol, 1,2-octanediol, 1,2-decanediol, ethylhexylglycerin, glyceryl caprate, glyceryl caprylate, glyceryl undecylenate, phenethyl alcohol, and phenylpropanol. The antimicrobial agents, whether used singly or as a blend of two or more antimicrobial agents, are to be used in the concentrations that vary from agent to agent and are to be introduced into the formulations in either organic or aqueous phase, all of which is known to those skilled in the art. Some compositions, such as those described in Examples 8 and 9, did not require the addition of anti-microbial agents to remain free of microbial contamination, even if left open to the atmosphere at ambient temperature for approximately six weeks, or in heat-sealed sachets for four approximately weeks. It is known that propylene glycol has antimicrobial activity at or above concentration of 20% and may potentiate the activity of other preservatives or exert its own antimicrobial activity at lower concentrations, however, the absence of microbial contamination without the addition of any antimicrobial preservatives was unexpected.

As used herein, the term “thickener” or “thickening agent” refers to substances, whether gelling or non-gelling, which raise viscosity and which may or may not require pH adjustment or addition of salts (ions) to produce increase in viscosity. Examples of “thickeners” or “thickening agents” are crosslinked polyacrylic acid polymers such as Carbopol® 71G, 940, 971P, 974P, 980, 981, 5984 EP, ETD 2020, Ultrez 10, Pemulen™ TR-1 and TR-2 NF polymers; hydroxyethyl acrylate/sodium acryloyldimethyl taurate copolymers; polyacrylate crosspolymer-6; sodium acrylate/acryloyldimethyltaurate/dimethylacrylamide crosspolymer; hyaluronic acid of average molecular weights of approximately 8,000-13,000, 50,000-75,000, 450,000-500,000, or one million or more Da; hydroxypropyl methylcellulose (HMPC, hypromellose, substitution types 2910, 2208, or 2906) in grades of viscosity of 2% aqueous solution of approximately 3 cP, 4 cP, 5 cP, 15 cP, 50 cP (40-60 cP), 100 cP (80-120 cP), 200-300 cP, 500-1000 cP, 1000-2000 cP, 4000 cP, methylcellulose, hydroxyethyl cellulose in grades of viscosity of 5% aqueous solution of 100 cP, 50-150 cP, of 2% aqueous solution at 20° C. of 200-300 cP, 800-1500 cP, approximately 2000 cP, approximately 3400 cP, or 5000 cP, ethylcellulose, hydroxypropyl cellulose, also gums such as xanthan gum, locust bean gum, guar gum, alginin, as well as agar gum, pectin, K-carrageenan, 1-carrageenan, as well as starches such as potato starch, corn (maize) starch, wheat starch, or pea starch. Some of the thickeners are multifunctional substances and in certain compositions a thickener may act as an anti-caking agent and/or a lubricating agent, and/or a humectant. As used herein, the term “lubricating agent” may refer to a thickener or it may refer to a substance that is not a thickener, for example, to lauric acid and its salts such as sodium laurate, or isopropyl myristate.

As used herein, the term “formulation” is used interchangeably with the term “composition.” In general, a “formulation” of the invention comprises one or more cannabinoids and phospholipids, and may contain one or more of surfactants, cryoprotectants, bulking agents, stabilizers, water-miscible solvents, anti-microbial agents, or thickeners. The compositions described herein are intended for use in pharmaceutical, phytopharmaceutical, nutraceutical, cosmetic, or veterinary settings by various routes of administration, such as dermal (topical or transdermal), mucosal (buccal, sublingual, gingival, vaginal, or rectal), or enteral (oral, ingestible) and may be formulated as an ointment, a cream, a suspension, a lotion, a paste, a gel, or a suppository, or in soft- or hard-shell capsules, or tinctures, or fluids of different viscosities, or serums, the basic preparation techniques of which are known to those skilled in the art.

As used herein, the term “application” or “applying”, or “administration”, or “administering” means placing or spreading or rubbing on a quantity of a composition to areas of skin, whether on face, neck, scalp, on extremities or torso, on or around external genitalia such as labia, or intravaginally, rectally, or onto an oral mucosa, whether buccal, or sublingual, or gingival, or may be ingested if formulated into or as food or beverage. When desirable, appropriate preservatives may be added, such as anti-microbial and anti-fungal agents or other agents as described above.

Formulation of the invention can be produced by a number of methods, including those described in the Examples and claims below.

The following examples are not intended to be limiting. Those of skill in the art will, in light of the present disclosure, appreciate that many changes can be made in the specific materials and which are disclosed and still obtain a like or similar result without departing from the spirit and scope of the invention.

### EXAMPLES

### Example 1. Preparation of THC-Loaded Sphingomyelin Pro-Liposomes

THC distillate containing 62.5% THC (71 mg, 44.4 mg THC) was dissolved in 10 mL of 3:2 mixture of TBA and water with mild warming (45° C. bath) and combined with a 90 mL solution of 400 mg sphingomyelin. This solution was frozen at −80° C. and freeze dried for 24 hours to obtain a low density, fluffy white powder.

### Example 2. Preparation of CBD-Loaded Phosphatidylcholine Proliposomes by Freeze Drying

A quantity of 600 mg of soya phosphatidylcholine was added to 130 mL of 3:2 mixture of TBA and water, and 67 mg of CBD was added to 20 mL of same mixture. The solids were dissolved by warming in a 50° C. water bath followed by sonication and combined to obtain a yellowish solution with lipid concentration of 4 mg/mL. The solution was split into four batches: Batch 1 contained 40 mL of SPC and CBD solution and no bulking agent; Batch 2 contained 40 mL of SPC and CBD solution plus 25 mg of PVP K16-18; Batch 3 contained 30 mL of SPC and CBD solution and 4 mg of TPGS; Batch 4 contained 35 mL of SPC and CBD solution and 28 mg of Soluplus. The batches were placed in freezer at −80° C. for one hour and placed on a freeze dryer. After 20 hours, Batches 1, 2, and 4 presented as white cakes while Batch 3 failed to produce a self-supporting cake and was a foamy semi-solid. Samples from batches 1, 2, and 4 were hydrated in deionized water at ambient temperature. Examination by optical microscopy revealed presence of large liposomes.

### Example 3. Preparation of CBD-Loaded Large Multilamellar Liposomes

A quantity of 200 mg of DPPC and 22 mg of CBD (10% w/w relative to lipid) were dissolved in 10 mL of TBA by warming in a water bath at 60° C. The resultant clear yellowish solution was maintained at −80° C. for 1.5 hours and freeze dried overnight to obtain a white, low density hygroscopic powder, which was hydrated with phosphate-buffered saline (PBS) at 60° C. Multilamellar liposomes were observed by optical microscopy.

### Example 4. Preparation of CBD-Loaded Large Multilamellar Liposomes

A quantity of 200 mg of SPC, 200 mg DSPC, and 25 mg of CBD (10% w/w relative to lipid) were dissolved in 10 mL of TBA by warming in a water bath at 60° C. The resultant clear yellowish solution was maintained at −80° C. for 1.5 hours and freeze dried overnight to obtain a white, low density, hygroscopic powder, which was hydrated with phosphate-buffered saline (PBS) at 60° C. Multilamellar liposomes were observed by optical microscopy.

### Example 5. Preparation of CBD-Loaded Phosphatidylcholine Proliposomes by Freeze Drying

SPC (5.14 g) and CBD (573 mg) were dissolved in TBA by warming in a water bath at 60° C. and equilibrated to just above ambient. This solution was slowly added to a solution of ascorbic acid (100 mg) and sodium ascorbate (450 mg) in 40 mL of deionized water (pH approximately 5) to obtain a yellow monophasic mixture. This mixture was concentrated on a rotary evaporator to obtain a white paste and to this was added a solution of 1.2 g of PVP K30, 100 mg ethyl cellulose, 150 mg sodium chloride in 25 mL of deionized water. The resultant mixture was frozen in dry ice/acetone bath over 30 minutes and freeze dried overnight to obtain a free-flowing, hygroscopic powder.

### Example 6. Preparation of CBD-Loaded DSPC Proliposomes by Freeze Drying

DSPC (1.5 grams) and CBD (160 mg) were dissolved in 20 mL TBA with mild warming. This solution was slowly added to a solution of citric acid (7.5 mg) and sodium ascorbate (90 mg) in 40 mL of deionized water (pH approximately 5) to obtain a suspension. Bulk solvent was removed by rotary evaporation to obtain a paste, which was frozen and freeze dried.

### Example 7. Preparation of CBD Liposome-Based Gel

A quantity of 2.7 g of powder obtained as described in Example 5 was hydrated with 10 mL deionized water with overhead stirring. A non-dripping gel was obtained by addition of Carbopol 940 and adjusting pH to 7 with triethanolamine.

### Example 8. One-Pot Preparation of CBD-Loaded PG Liposomes and Evaluation of its Microbial Contamination on Storage

Hydrogenated soya phosphatidylcholine (2.7 grams) and CBD (0.3 grams) were dissolved in propylene glycol (3.5 mL) in a closed vessel by heating in a water bath at approximately 70° C. with magnetic stirring. This solution was added quickly, with magnetic stirring, to a solution of citric acid (27 mg) and sodium ascorbate (280 mg) in 29 mL of deionized water pre-warmed in a water bath (65° C. bath temperature) to form a white suspension. The suspension was stirred at 65° C. bath temperature for approximately one hour then removed from heat with continued stirring.

The resultant formulation was covered with aluminum foil and stored at ambient temperature for approximately six weeks, whereupon a sample was removed and tested for microbial contamination using the industry accepted methods based on the Biomérieux TEMPO® system (Cirolini, A., et al. (2013). “Evaluation of the Petrifilm™ and TEMPO® systems and the conventional method for counting microorganisms in pasteurized milk.” Food Science and Technology 33: 784-789) and the BioRad QPCR platform (Nde, C. W., et al. (2008). “An Evaluation of Conventional Culture, invA PCR, and the Real-Time PCR iQ-Check Kit as Detection Tools for Salmonella in Naturally Contaminated Premarket and Retail Turkey.” Journal of Food Protection 71(2): 386-391). The BioRad platform was used to analyze for the presence of Shiga toxin-producing E. coli (STEC) and salmonella, and the TEMPO® system for others. The results are shown in the Table below.

| | Allowable Limit | Amount Detected | Disposition |
|---|---|---|---|
| Total Viable Aerobic Bacteria | 100000 CFU/g | <100 CFU/g | Pass |
| Total Yeast & Mold | 10000 CFU/g | <100 CFU/g | Pass |
| Total Coliform Bacteria | 1000 CFU/g | <100 CFU/g | Pass |
| Total Bile-Tolerant Gram | 1000 CFU/g | <100 CFU/g | Pass |
| Negative Bacteria | | | |
| STEC | Detected in 1 gram | Negative | Pass |
| Salmonella | Detected in 1 gram | Negative | Pass |

### Example 9. One-Pot Preparation of CBD-Loaded PG Liposomes

Hydrogenated soya phosphatidylcholine (5.4 grams) and CBD (0.6 grams) were dissolved in propylene glycol (6 mL) in a closed vessel by heating in a water bath at approximately 85° C. with magnetic stirring. This solution was added quickly, with overhead stirring, to a solution of citric acid (54 mg) and sodium ascorbate (600 mg) in 50 mL of deionized water pre-warmed in a water bath at 60° C. to form a white suspension. The suspension was stirred at 60° C. bath temperature for approximately one hour then removed from heat with continued stirring.

### Example 10. One-Pot Preparation of Lotion of CBD-Loaded PG Liposomes

Hydrogenated soya phosphatidylcholine (10.8 grams) and CBD (1.2 grams) were dissolved in propylene glycol (12 mL) by heating in a water bath at 80-90° C. with magnetic stirring. This solution was added, with overhead stirring, over approximately 30 seconds to a solution of ascorbic acid (50 mg) and sodium ascorbate (500 mg) in 100 mL of deionized water pre-warmed in a water bath at 65° C. to form a white suspension. The suspension was stirred at 65° C. bath temperature for approximately 30 minutes then removed from heat with continued stirring. To a warm suspension, with continued stirring, was added 300 mg (0.25% w/w) polyacrylate crosspolymer-6 to form a white lotion.

Hydrogenated soya phosphatidylcholine (5.4 grams) was dissolved in in propylene glycol (6 mL) by heating in a water bath at approximately 85° C. with overhead stirring. To this solution CBD (0.6 grams) was added until a clear yellowish mixture was formed. A solution of ascorbic acid (25 mg) and sodium ascorbate (250 mg) in 50 mL of deionized water was pre-warmed in a water bath at 65° C. to and this aqueous buffer was added to the propylene glycol mixture quickly (approximately 30 seconds) with overhead stirring to form a white suspension. This mixture was stirred at 65° C. bath temperature for approximately 15 minutes and to it was added polyacrylate crosspolymer-6 (75 mg, 0.1% w/w) to form a white lotion. The water bath was removed and the formulation was stirred while cooling to ambient temperature.

### Example 11. Microbial Contamination Analysis of Sachets Filled with Lotion of CBD-Loaded PG Liposomes

The lotion produced as described in Example 10 was loaded into sachets, 2 grams of lotion per sachet, and the sachets were heat sealed. After four weeks of storage at ambient temperature, the contents were removed and tested for microbial contamination using the industry accepted methods based on the Biomérieux TEMPO® system (Cirolini, A., et al. (2013). “Evaluation of the Petrifilm™ and TEMPO® systems and the conventional method for counting microorganisms in pasteurized milk.” Food Science and Technology 33: 784-789) and the BioRad QPCR platform (Nde, C. W., et al. (2008). “An Evaluation of Conventional Culture, invA PCR, and the Real-Time PCR iQ-Check Kit as Detection Tools for Salmonella in Naturally Contaminated Premarket and Retail Turkey.” Journal of Food Protection 71(2): 386-391). The BioRad platform was used to analyze for the presence of Shiga toxin-producing E. coli (STEC) and salmonella, and the TEMPO® system for others. The results are shown in the Table below.

| | Allowable Limit | Amount Detected | Disposition |
|---|---|---|---|
| Total Viable Aerobic Bacteria | 100000 CFU/g | <100 CFU/g | Pass |
| Total Yeast & Mold | 10000 CFU/g | <100 CFU/g | Pass |
| Total Coliform Bacteria | 1000 CFU/g | <100 CFU/g | Pass |
| Total Bile-Tolerant Gram | 1000 CFU/g | <100 CFU/g | Pass |
| Negative Bacteria | | | |
| STEC | Detected in 1 gram | Negative | Pass |
| Salmonella | Detected in 1 gram | Negative | Pass |

### Example 12. One Pot Preparation of Lotion of CBD-Loaded PG Liposomes

Hydrogenated soya phosphatidylcholine (10.8 grams) and CBD (1.2 grams) were dissolved in propylene glycol (12 mL) by heating in a water bath at 80-90° C. with magnetic stirring. This solution was added, with overhead stirring, over approximately 30 seconds to a solution of ascorbic acid (50 mg) and sodium ascorbate (500 mg) in 100 mL of deionized water pre-warmed in a water bath at 65° C. to form a white suspension. The suspension was stirred at 65° C. bath temperature for approximately 30 minutes then removed from heat with continued stirring. To a warm suspension, with continued stirring, was added 300 mg (0.25% w/w) polyacrylate crosspolymer-6 to form a white lotion.

### Example 13. One-Pot Preparation of CBD-Loaded Glycerosomes

Hydrogenated soya phosphatidylcholine (2.7 grams), CBD (0.3 grams), and polyethylene glycol monostearate (50 mg) were mixed with glycerin (3 mL) and deionized water (27 mL) containing 25 mg citric acid and 275 mg sodium ascorbate, and heated in a water bath at 80° C. with magnetic stirring until a homogenous suspension was formed. Evaluation by optical microscopy revealed presence of a mixture of round vesicles and vesicle aggregates approximately 1-10 μm in size.

### Example 14. One-Pot Preparation of CBD-Loaded Liposomes (Failed)

Hydrogenated soya phosphatidylcholine (2.7 grams), CBD (0.3 grams), and polyethylene glycol monostearate (50 mg) were mixed with deionized water (30 mL) containing 25 mg citric acid and 275 mg sodium ascorbate, and heated in a water bath at 80° C. with magnetic stirring until a homogenous suspension was formed. Examination by optical microscopy revealed presence of a mixture of round vesicles and droplets approximately 2-20 μm in size. On equilibration to ambient temperature, oily slicks appeared on surface indicating that CBD encapsulation in liposomal vesicles was incomplete in absence of glycerin (compare with Example 13).

### Example 15. Condom Compatibility Testing

Condom compatibility testing was performed following the general principles of the American Society for Materials Testing. Copious quantity of liposomal suspension prepared as described in Example 8 was slathered onto a stretched latex condom (Duree), which maintained its tensile strength. Another condom sample was treated similarly and expanded into a balloon of approximately 1×0.5 feet (air burst test) and did not rupture.

Controls: exposure of a stretched latex condom (Durex®) to TBA had no effect; exposure of a stretched latex condom (Durex®) to a 10 mg/mL solution of CBD in TBA caused instant rupture.

1. A “one-pot” method for the preparation of a suspension formulation of one or more cannabinoid(s) in large liposomes, said method comprising: (a) the addition of a pre-heated homogenous mixture of phospholipids, and cannabinoid(s) in a water-miscible solvent to a pre-heated aqueous solution under stirring; or (b) the addition of a pre-heated aqueous solution to a pre-heated homogenous mixture of phospholipids, and cannabinoid(s) in a water-miscible solvent, wherein both (a) or (b) are followed by stirring in a manner and for a period of time such that a homogenous suspension is produced, wherein the formation of the suspension is followed by the addition of a thickening and/or a lubricating agent, thereby producing a resultant formulation.

2. (canceled)

3. The method of claim 1, wherein the water-miscible solvent is propylene glycol.

4. The method of claim 3, wherein the resultant formulation contains no more than 20% of propylene glycol on w/w basis.

5. The method of claim 4, wherein the resultant formulation contains 8-12% of propylene glycol on w/w basis.

6. The method of claim 1, wherein the water-miscible solvent is glycerin.

7. The method of claim 6, wherein the resultant formulation contains no more than 20% of glycerin on w/w basis.

8. The method of claim 7, wherein the resultant formulation contains 8-12% of glycerin on w/w basis.

9. The method of claim 1, wherein the water-miscible solvent is a mixture of propylene glycol and glycerin.

10. The method of claim 9, wherein the resultant formulation contains no more than 20% of propylene glycol and glycerin on a w/w basis.

11. The method of claim 1, wherein the resultant formulation comprises stabilizers which are added to it at any step before or after obtaining the formulation.

12. The method of claim 11, wherein the stabilizers are one or both of ascorbic acid and sodium ascorbate.

13. The method of claim 1, wherein the resultant formulation comprises preservatives which are added to it at any step before or after obtaining the resultant formulation.

14. (canceled)

15. The method of claim 1, wherein the cannabinoid(s) is/are selected from the group consisting of Δ⁹-THC, Δ⁸-THC, CBD, and CBN.

16. The method of claim 15, wherein the cannabinoid is CBD.

17. The method of claim 15, wherein the cannabinoid is THC.

18. The method of claim 15, wherein the cannabinoids are CBD and THC.

19. The method of claim 1, wherein the thickening agent is polyacrylate crosspolymer-6.

20. The method of claim 1, wherein the phospholipids are selected from SPC, HSPC, and DSPC.

21. The method of claim 1, wherein the lubricating agent is hydroxyethyl cellulose.

22. The resultant formulation produced by the method of claim 1.

23. The formulation of claim 22, wherein the formulation is compatible with polyurethane, latex and polyisoprene condoms.

24. A condom for use in sexual activity, wherein the condom is pre-coated with a formulation of claim 22.

25. The formulation produced by claim 1, wherein the formulation comprises HSPC, one or more cannabinoid(s), ascorbic acid, sodium ascorbate, propylene glycol, polyacrylate crosspolymer-6, and water.

26. The formulation of claim 25, comprising preservatives.

27. The formulation produced by claim 1, wherein the formulation comprises HSPC, one of more cannabinoids, ascorbic acid, sodium ascorbate, propylene glycol, hydroxyethyl cellulose, hyaluronic acid, and water.

28. The formulation of claim 27, comprising preservatives.

29. The formulation of claim 1, wherein the cannabinoid(s) is/are selected from the group consisting of Δ⁹-THC, Δ⁸-THC, CBD, and CBN.

30. The formulation of claim 29, wherein the cannabinoid(s) is/are one or both of THC and CBD.

31. The formulation of claim 30, wherein the cannabinoid is CBD.

32. A liposomal formulation, comprising HSPC, one or more cannabinoid(s), ascorbic acid, sodium ascorbate, propylene glycol, polyacrylate crosspolymer-6, and water, wherein the liposomal formulation is in the form of homogeneous suspension.

33. The formulation of claim 32, comprising preservatives.

34. A liposomal formulation, comprising HSPC, one or more cannabinoids, ascorbic acid, sodium ascorbate, propylene glycol, hydroxyethyl cellulose, hyaluronic acid, and water, wherein the liposomal formulation is in the form of a homogeneous suspension.

35. The formulation of claim 34, comprising preservatives.

36. (canceled)

37. The formulation of claim 29, wherein the cannabinoid(s) is/are one or both of THC and CBD.

38. The formulation of claim 37, wherein the cannabinoid is CBD only.

39. A formulation of one or more cannabinoid(s) in proliposomes comprising phospholipids and one or more cryoprotectants.

40. A formulation of claim 39, further comprising stabilizers.

41. (canceled)

42. A formulation of claim 39, wherein the phospholipid(s) is/are selected from the group consisting of EPC, SPC, and HSPC.

43. A formulation of claim 39, wherein the cryoprotectants(s) is/are selected from the group consisting of PVP, hydroxypropyl-β-cyclodextrin, inulin, or HPMC.

44. A formulation of claim 40, wherein the stabilizers(s) is/are selected from the group consisting of citric acid, sodium citrate, ascorbic acid, and sodium ascorbate.

45. A liposomal formulation produced by hydration of the proliposomes of claim 39 with water or aqueous buffer.
